(19) United States
(12) Patent Application Publication
(10) Pub. No.: US 2020/0352772 A1
(43) Pub. Date: Nov. 12, 2020

(54) DEVICE FOR ALLEVIATING BACK TENSION OR PAIN

(71) Applicant: Joseph CAIOZZO, Sarasota, FL (US)

(72) Inventor: Joseph CAIOZZO, Sarasota, FL (US)

(21) Appl. No.: 16/403,828

(22) Filed: May 6, 2019

(51) Int. Cl. A61F 5/02

(52) U.S. Cl. CPC A61F 5/028; A61F 5/30

(57) ABSTRACT

A device for alleviating back tension or pain includes a flexible fabric support configured in an operable arrangement to be draped over a rear central portion of a belt worn by a user with an inner surface facing the belt. At least one pocket is formed at a lower portion of an outer surface of the flexible fabric support. A plurality of pressure elements is disposed within the pocket, each pressure element including a curved hollow body having a convex surface with a flattened portion facing the user's back and a concave surface facing away from the user's back in the operable arrangement. A foam layer covers at least a portion of the convex surface and the flattened portion. In the operable arrangement, the pressure elements press firmly against the user's erector spine muscles in a lumbar region on both sides of the spine.

### BACKGROUND OF THE INVENTION

### 1. Field of the Invention

The invention relates to devices for alleviating back tension or pain. In particular, the invention relates to devices configured to be used with conventional back support belts which devices include pressure elements adapted to press firmly against a user's erector spine muscles in a lumbar region on both sides of the spine.

### 2. The Prior Art

Various support belts are known for support of the muscles of a person's back. Most of the previous back belts suggest using a soft material on the various positions along the back. Additionally, many of the known support devices prohibit or impede a user's mobility or ability to participate in active movement when using the device.

### SUMMARY OF THE INVENTION

The crux of this invention is to apply pressure against the two main erector spine muscles, the abductor and extensor muscles, that run parallel to the spiney bone down the center of the back. A basic principle is that when your back is under strain or is damaged so that the nerves coming out of the spinal column are pressed or irritated, the nerves send a pain signal to the adjacent muscles; these muscles spasm and/or contract and send a pain signal. A device according to an embodiment of the invention, contrary to many of the conventional devices, proposes to use two or more pressure bars of specific characteristics (explained separately herein) and press them tightly against the major muscles of the back.

Pressing the muscles firmly, contrary to conventional opinion, will actually ease the tension of the muscle that has contracted. This mechanism is much like the numerous devices utilized on the arm that press firmly against the ulna nerve and actually relieve the condition commonly referred to a tennis elbow.

Another unique feature of a device according to an embodiment of the invention is its portability. There are approximately 75 million Americans who suffer from some form of back pain or ailment. There are likewise millions of back belts used of various size and construction; from air belts to leather belts. This device can be successfully incorporated into any one of the many belts already used by millions of people.

A device according to an embodiment of the invention can be simply folded or placed over the back of an existing belt or back support with the pressure bars facing the wearer's back. The user then secures the existing belt as it is designed with a slip, velcro, belt buckle or fastener.

A device for alleviating back tension or pain according to an aspect of the invention includes a flexible fabric support having an inner surface and an outer surface. The flexible fabric support is configured in an operable arrangement to be draped over a rear central portion of a belt worn by a user with the inner surface of the flexible fabric support facing the belt. At least one pocket is formed at a lower portion of the outer surface of the flexible fabric support and a plurality of pressure elements are disposed within the at least one pocket. Each of the pressure elements include a curved hollow body having a convex surface with a flattened portion facing the user's back in the operable arrangement and a concave surface facing away from the user's back in the operable arrangement. A foam layer covers at least a portion of the convex surface and flattened portion of the pressure element. The pressure elements press firmly against the user's erector spine muscles in a lumbar region on both sides of the spine in the operable arrangement.

In a further aspect of the invention, the pressure elements include beveled end portions, such as for example 45 degree bevels.

In a further aspect of the invention, hook and loop fastening elements are disposed on the inner surface of the flexible fabric support.

In a further aspect of the invention, the at least one pocket includes multiple pockets and each of the pressure elements is disposed within a single respective pocket of the multiple pockets.

In a further aspect of the invention, the at least one pocket is a single pocket and the pressure elements are disposed within the single pocket.

In further aspects of the invention, the flexible fabric support is constructed from a 300 denier polyester material or a canvas material.

In a further aspect of the invention, a plastic coating covers at least a portion of the flexible fabric support.

In a further aspect of the invention, the foam layer includes an ethylene vinyl acetate foam.

In a further aspect of the invention, the foam layer is in the range of approximately 0.125 to 0.250 inches in thickness.

In a further aspect of the invention, the flattened portion of the pressure elements is approximately 0.250 inches in height.

In a further aspect of the invention, the pressure elements have a minimum thickness of 0.113 inches.

In further aspects of the invention, the pressure elements have a collapse pressure of at least 1000 psi and/or a tensile strength of at least 264 psi.

The pressure bars are designed specifically for use in the lumbar region specifically because it is the area of the back most frequently injured or in pain and is perfectly suited for this application.

An advantage of the pressure bars is that when the back muscle becomes tight from use or exertion, the pressure bars may allow continued performance of such activity or exercise. For example, if an individual playing golf feels a twinge when making a golf swing, the pressure bars will replace the therapist massage by pressing the bars with the attendant back belt into the muscle. By maintaining pressure on both lumbar muscles simultaneously, movement will be allowed to continue.

Most of the previous back belts suggest using a soft material on the various positions along the back. The inventive arrangement runs contrary to previous back belts because instead of a softer cushion, with the proposed hard structure of the pressure bar, only covered by a piece of foam board to minimize friction, the benefit will within a few minutes allow the user to continue their activity by reducing the spasm with pressure. This arrangement will not treat or cure the condition, but will suspend the limitations of the back muscle spasm to allow continued activity.

Another distinct feature of the disclosed pressure bar is it will stay pressed against the muscle regardless of position or movement. Because of the specific size and arrangement, size and arrangement, the pressure bars encompass both muscles simultaneously and remain pressed against the two muscles whether the user turns, bends or just walks. The specialty of the pressure bars is for the lumbar region.

### BRIEF DESCRIPTION OF THE DRAWINGS

Other benefits and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a device for alleviating back tension or pain according to an embodiment of the invention in a folded arrangement for use with a conventional back support belt;

FIG. 2 shows a cross sectional view of the device according to FIG. 1;

FIG. 3 shows a view of a device according to another embodiment of the invention; and

FIG. 4 shows a cross sectional view of a portion of a device according to an embodiment of the invention.

### DETAILED DESCRIPTION OF THE DRAWINGS

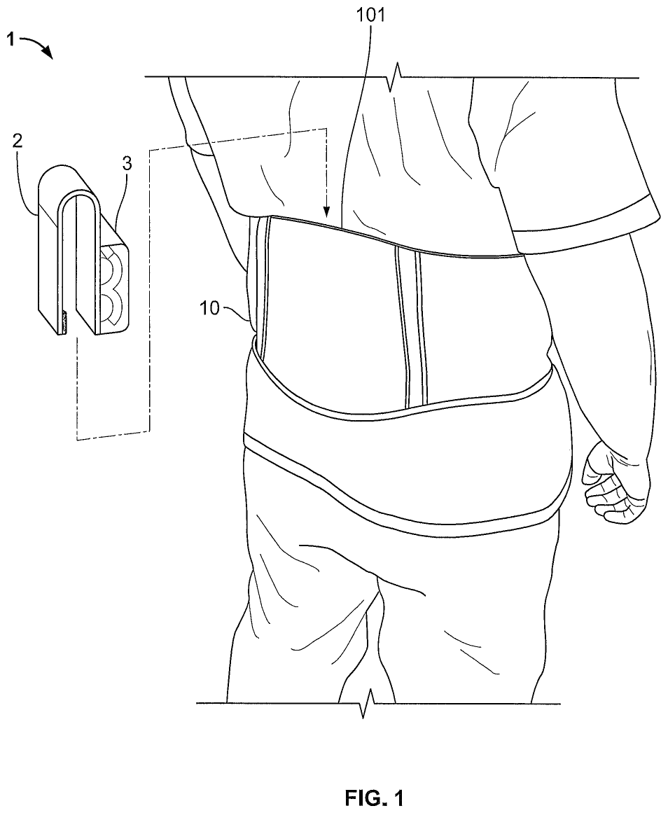

Referring now in detail to the drawings and, in particular, FIG. 1 shows a device 1 for alleviating back tension or pain according to an embodiment of the invention. the device 1 includes a flexible fabric support or sleeve 2 having an inner surface 21 and an outer surface 22. The flexible fabric support 2 is configured in an operable arrangement to be draped over a rear central portion 101 of a belt 10, such as for example a conventional back support belt, with the inner surface 21 of the flexible fabric support 2 facing the belt 10.

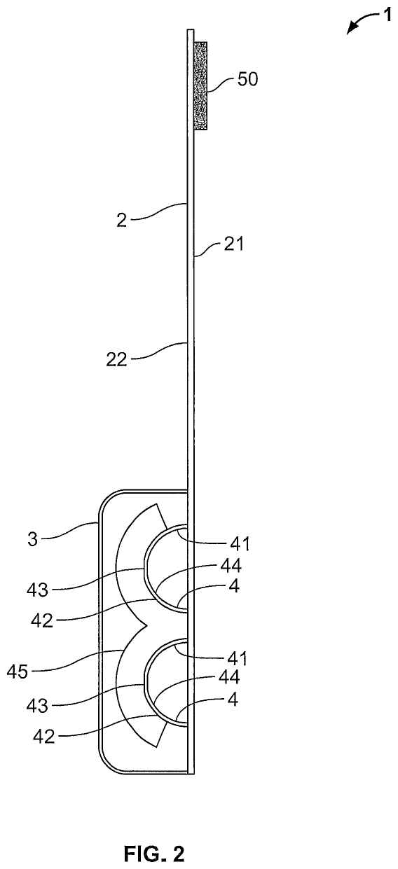

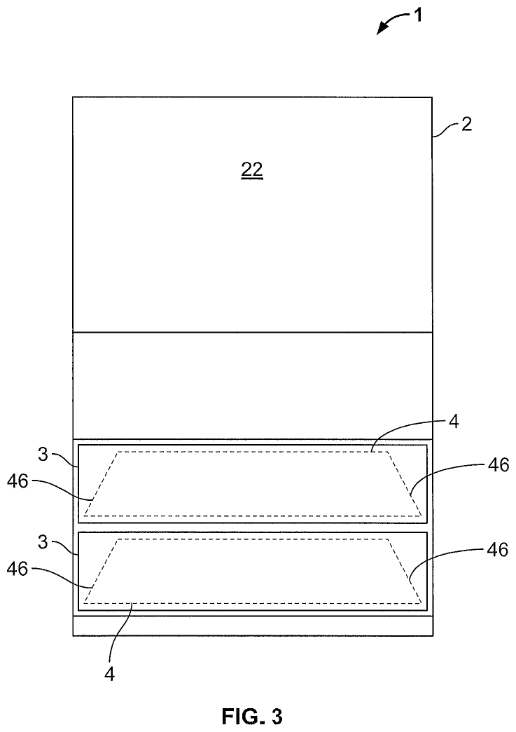

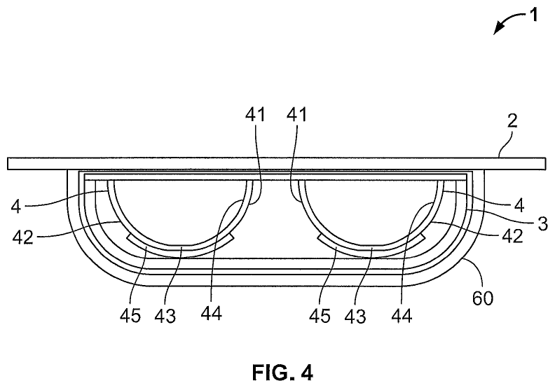

As shown in FIGS. 2, 3 and 4, at least one pocket 3 is formed at a lower portion of the outer surface 22 of the flexible fabric support 2 and a plurality of pressure elements 4 is disposed within the at least one pocket 3. The pressure elements 4 may be disposed in respective pockets 3 or a single pocket 3 of the flexible fabric support 2. In other words, the at least one pocket 3 may include a plurality of pockets 3 and each of the plurality of pressure elements 4 may be disposed within a single respective pocket 3 of this plurality of pockets 3 (as shown in FIG. 3) or the at least one pocket 3 may be a single pocket 3 with multiple pressure elements 4 disposed within the single pocket 3 (as shown in FIG. 2).

Each of the pressure elements includes a curved hollow body 41 having a convex surface 42 with a flattened portion 43 facing the user's back in the operable arrangement and a concave surface 44 facing away from the user's back in the operable arrangement.

For example, a top portion of the semi-circular pressure element 4 may be machined or cut so as to present a flattened portion or surface 43 approximately ¼ inch in width against the back muscle. Thus, a portion of the convex surface 42 of the pressure element facing the back includes a flattened region 43, for example a flat portion having a minimum width of approximately ¼ inch.

As shown in FIGS. 2 and 3, a foam layer 45 covers at least a portion of the convex surface 42 and flattened portion 43 of the pressure elements 4. In particular, the pressure elements 4 may be partially encased in a foam layer 45 to minimize the back to pressure element hardness, as it is imperative that the pressure elements 4 press firmly into the user's two major back muscles. The foam for this purpose may be, for example EVA (ethylene vinyl acetate) foam of approximately ⅛-¼ inch in thickness.

The pressure elements 4 press firmly against the user's erector spine muscles in a lumbar region on both sides of the spine in the operable arrangement.

The flexible fabric support or sleeve 2 that houses the pressure elements 4 may be, for example, approximately 13-13½ inches long by 6¾-7 inches wide with two pockets 4 approximately one inch wide and closed at the ends to support the pressure elements 4. The fabric support or sleeve 2 sleeve can be made of a flexible fabric or fiber, for example, 300 denier polyester or canvas and is lightweight with both strength and flexibility.

For an average person of approximately five feet ten inches in height and weighing approximately 185 pounds, the pressure elements may be, for example 6 inches long with a tensile strength of not less than 264 pounds and a collapse pressure of at least approximately 1000 psi. The pressure elements, for a standard male of approximately five foot ten inches to six feet weighing approximately 170-200 pounds may measure 6 inches in length.

The pressure elements 4 may have a bevel 46 at their ends to mesh into the existing belt 10 with a minimum of interference and to retain a desired length of the pressure bar pressed against the back and present a contour for the back belt during use. For example the pressure bars may be cut or otherwise provided with a 45 degree angle beveled end portions 46 and maintain a 6½ inch length of the pressure element 4 pressing against the back.

The length of the pressure elements 4 may vary in ½ inch increments from small to extra large.

A thin plastic coating 60 may be provided on the inside of the fabric support 2 so the absorption of perspiration (moisture) is slowed down slightly. The fabric support 2 may not be waterproof (as a waterproof material may be prohibitively heavy for the application) and plastic is not comfortable, but the coating 60 functions to make the detachable support 2 more comfortable.

As described above, the flexible fabric support 2 is configured to be inserted between a person's existing belt 10 and their back, with the pressure elements 4 pressed against the back and a portion of the support 2 then flapped or draped over the outside of the existing belt 10. The flexible fabric support 2 may have a hook and loop type fastening element or tab 50, such as a male hook and loop fastening tab suitable for attaching to most fabric belts. A female or opposite hook and loop fastener can be included with the pressure element portion of the support 2 so as to extend the suitability of the device to as many existing belts as possible. The hook and loop fastening elements may be disposed on the inner surface 21 of the flexible fabric support 2.

The pressure bars may have a minimum wall thickness of approximately 0.113 inches, a tensile strength not less than 264 pounds and a minimum collapse strength of 1,000 psi—sufficient to allow maximum pressure against the back muscles.

Accordingly, while a number of embodiments of the present invention have been shown and described, it is obvious that many changes and modifications may be made thereunto without departing from the spirit and scope of the invention.

### REFERENCE NUMERALS

1 device for alleviating back tension or pain
2 flexible fabric support
3 pocket
4 pressure elements
10 belt
21 inner surface
22 outer surface
41 curved hollow body
42 convex surface
43 flattened portion
44 concave surface
45 foam layer
46 beveled end portions
50 hook and loop fastening elements
60 plastic coating
101 rear central portion of belt

What is claimed is:

1. A device for alleviating back tension or pain, the device comprising:
a flexible fabric support having an inner surface and an outer surface, said flexible fabric support configured in an operable arrangement to be draped over a rear central portion of a belt worn by a user with said inner surface facing the belt;
at least one pocket formed at a lower portion of said outer surface of said flexible fabric support;
a plurality of pressure elements disposed within said at least one pocket, each of said pressure elements comprising a curved hollow body having a convex surface with a flattened portion facing the user's back in the operable arrangement and a concave surface facing away from the user's back in the operable arrangement; and
a foam layer covering at least a portion of said convex surface and said flattened portion of said pressure elements;
wherein said plurality of pressure elements press firmly against the user's erector spine muscles in a lumbar region on both sides of the spine in the operable arrangement.

2. The device for alleviating back tension or pain according to claim 1, wherein said pressure elements comprise beveled end portions.

3. The device for alleviating back tension or pain according to claim 2, wherein said beveled end portions comprise 45 degree bevels.

4. The device for alleviating back tension or pain according to claim 1, further comprising hook and loop fastening elements disposed on said inner surface of said flexible fabric support.

5. The device for alleviating back tension or pain according to claim 1, wherein said at least one pocket comprises a plurality of pockets and each of said plurality of pressure elements is disposed within a single respective pocket of said plurality of pockets.

6. The device for alleviating back tension or pain according to claim 1, wherein said at least one pocket comprises a single pocket and said pressure elements are disposed within said single pocket.

7. The device for alleviating back tension or pain according to claim 1, wherein said flexible fabric support comprises a 300 denier polyester material.

8. The device for alleviating back tension or pain according to claim 1, wherein said flexible fabric support comprises a canvas material.

9. The device for alleviating back tension or pain according to claim 1, further comprising a plastic coating over at least a portion of said flexible fabric support.

10. The device for alleviating back tension or pain according to claim 1, wherein said foam layer comprises an ethylene vinyl acetate foam.

11. The device for alleviating back tension or pain according to claim 1, wherein said foam layer is in the range of approximately 0.125 to 0.250 inches in thickness.

12. The device for alleviating back tension or pain according to claim 1, wherein said flattened portion is approximately 0.250 inches in height.

13. The device for alleviating back tension or pain according to claim 1, wherein said pressure elements have a minimum thickness of 0.113 inches.

14. The device for alleviating back tension or pain according to claim 1, wherein said pressure elements have a collapse pressure of at least 1000 psi.

15. The device for alleviating back tension or pain according to claim 1, wherein said pressure elements have a tensile strength of at least 264 psi.
